(19) United States
(12) Patent Application Publication
(10) Pub. No.: US 2019/0178867 A1
(43) Pub. Date: Jun. 13, 2019

(54) Method for Identification of Tissue Objects in IHC Without Specific Staining

(71) Applicant: Flagship Biosciences, Inc., Westminster, CO (US)

(72) Inventors: Cris L Luengo Hendriks, Broomfield, CO (US); Roberto Gianani, Lakewood, CO (US)

(73) Assignee: Flagship Biosciences, Inc., Westminster, CO (US)

(21) Appl. No.: 16/271,525

(22) Filed: Feb. 8, 2019

Related U.S. Application Data
(63) Continuation-in-part of application No. 15/396,552, filed on Dec. 31, 2016.

(51) Int. Cl. G01N 33/483; G01N 1/30; G01N 33/52; G01N 21/64

(52) U.S. Cl. CPC G01N 33/4833; G01N 1/30; G01N 2021/6441; G01N 21/6428; G01N 33/52

(57) ABSTRACT

The present invention concerns detection of specific tissue objects within thin sections of tissue samples as imaged in a brightfield microscope, such as a predetermined type of immune cells, without using a chromogenic stain that is specific to those tissue objects. The invention uses fluorescent stain and fluorescence imaging to detect these tissue objects. By combining a brightfield and a fluorescence image of the same tissue sample, it is possible to automatically identify objects in the brightfield image that have been stained for in the fluorescence image. The fluorescence stain does not affect the appearance of the tissue sample under the brightfield microscope. Therefore, the invention is ideal to automate the collection of training data for machine learning systems that are to be trained to detect these specific tissue objects in brightfield images of tissue that has not been stained to specifically highlight these tissue objects.

### CROSS-REFERENCE TO RELATED APPLICATIONS

This application is a continuation-in-part (CIP) of U.S. Ser. No. 15/396,552, filed Dec. 31, 2016, and titled “METHODS FOR DETECTING AND QUANTIFYING MULTIPLE STAINS ON TISSUE SECTIONS”;

the contents of which are hereby incorporated by reference.

### BACKGROUND

### Field of the Invention

The present invention relates generally to image analysis methods for the assessment of tissue samples. More specifically, the present invention relates to image analysis methods for the evaluation of tissue objects within a tissue sample without directly staining for those tissue objects.

Tissue samples are generally preserved, embedded in a block of paraffin, cut into thin sections, and one section placed on a glass slide. This section is then further prepared and stained for viewing. Stains can be either chromogenic or fluorescent, which are visible in a brightfield and a fluorescence microscope, respectively. The staining aids in viewing the tissue slide, which otherwise is so thin as to be nearly transparent, but can also be used to tag specific components of the tissue, for example highlighting cells that express a specific protein.

It is difficult to use many different stains. With a brightfield microscope, we are typically limited to two or three chromogenic stains (not only because of the difficulty of combining the stains, but mostly because of the difficulty of distinguishing the colors on the slide). In digital imaging, using an RGB camera, no more than three stains can be separated consistently. This limit is flexible if stains are not collocated, or if using more complex imaging methods.

With a fluorescence microscope it is easier to distinguish more dyes, because fluorescent dyes can be separated by their wavelengths. However, fluorescence staining and imaging has other problems that make it less suitable for pathology in the clinic, such as reduced stain longevity compared to chromogenic staining.

Reducing the number of stains used for an assay makes the assay less expensive, easier to use in the clinic, and more robust. There is a struggle in the industry to reduce the total number of stains in an assay while still increasing the amount of information gathered from the assay.

### Description of the Related Art

Tissue preparation and staining is well established in the field, and of common knowledge to one of ordinary skill in the art. Typical methods for tissue staining involve using either chromogenic or fluorescent stains on a single tissue section.

Both brightfield and fluorescent imaging is well understood by one of ordinary skill in the art. It is standard practice to use high-powered fields when using traditional microscopy and whole slide imaging with a digital pathology workflow.

Many methods are proposed in the literature for image alignment or registration, which can be broadly separated into rigid and elastic methods. Rigid registration allows only for a rotation and translation to match one image to the other, whereas elastic registration deforms one of the images to match the other. For alignment of whole-slide images of consecutive tissue sections, elastic registration methods are commonly applied. Rigid registration is not favored in the art as it typically does not provide the same quality of alignment compared to an elastic alignment for consecutive tissue sections.

As an alternative to aligning two images based on their individual pixels, it is possible to detect, for example, cells in both images, then align the cells based on their relative positions. This can be accomplished with efficient point cloud registration algorithms that have been developed primarily within the robot vision field. This registration process leads to a one-to-one assignment of cells in one image to cells in the other image. This assignment can then be used to transfer cell information obtained in one image to cells in the other image, or it can be used to derive a rigid or elastic transformation of one image to match the other. However, in the industry, as alignment is typically used for images of multiple tissue sections, the transfer of information is limited in scope, as the distance between tissue sections is such that at most a small fraction of cells will show in both sections. This lack of correspondences can make this method very inaccurate for consecutive sections, and useless if the distance between sections increases.

### SUMMARY

In accordance with the embodiments herein, a method for detecting and identifying tissue objects without direct staining is disclosed. The method described herein generally utilizes digital image analysis of a pair of images, one brightfield and one fluorescence, of a stained tissue section. The tissue section must be stained with a chromogenic stain and a fluorescent stain that stains tissue objects, such as immune active cell clusters, imaging the stained tissue section in both brightfield and fluorescence image modalities, aligning the digital images using any of number of image alignment techniques, and analyzing the aligned images, such that staining from the fluorescence image can be used to identify cells within the brightfield image.

### BRIEF DESCRIPTION OF THE DRAWINGS

FIG. 1 provides a general overview of the general method of the invention.

FIG. 2 provides an overview of an alternate embodiment of the invention.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

For purpose of definition, a tissue object is one or more of a cell (e.g., immune cell), cell sub-compartment (e.g., nucleus, cytoplasm, membrane, organelle), cell neighborhood, tissue compartment (e.g., tumor, tumor microenvironment (TME), stroma, lymphoid follicle, healthy tissue), blood vessel, and lymphatic vessel. Tissue objects are visualized by histologic stains which highlight the presence and localization of the tissue object. Tissue objects can be identified directly by stains specifically applied to highlight that tissue object (e.g., hematoxylin to visualize nuclei, IHC stain for a protein specifically found in a muscle fiber membrane), indirectly by stains applied which non-specifically highlight the tissue compartment (e.g., DAB staining), or are biomarkers known to be localized to a specific tissue compartment (e.g., nuclear-expressed protein, carbohydrates only found in the cell membrane).

For the purpose of definition, patient status includes diagnosis of disease state, disease severity, disease progression, and therapy efficacy. Other patient statuses are contemplated.

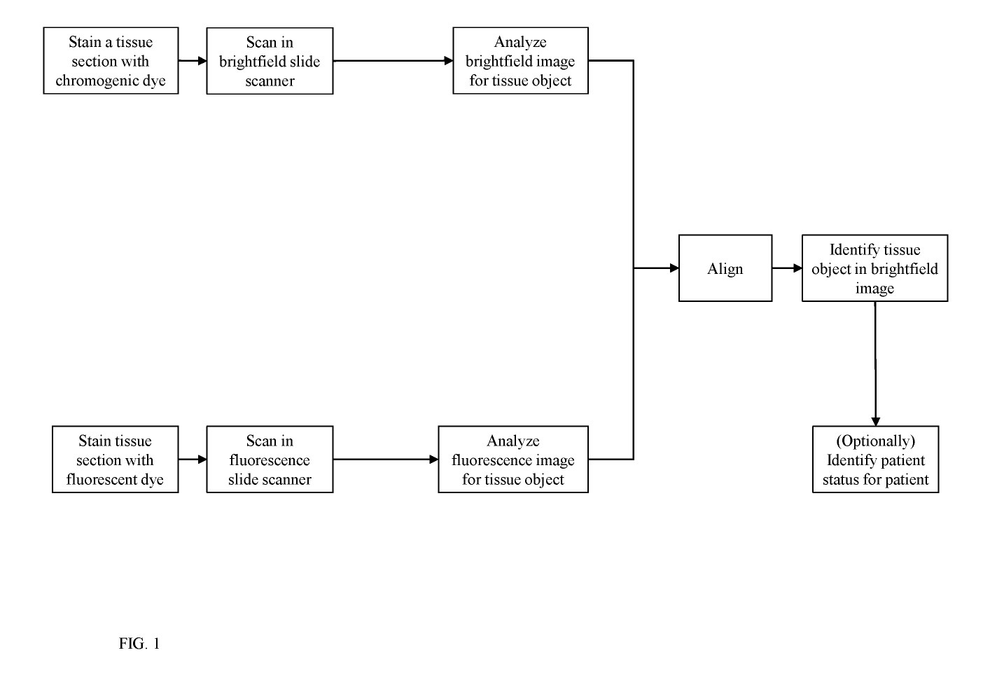

In an illustrative embodiment of the invention, as summarized in FIG. 1, the method may be summarized of the following seven steps: i) staining a tissue section with a chromogenic dye; ii) staining the same tissue section with a fluorescent dye that stains for a specific type of tissue object; iii) acquiring a brightfield digital image of the tissue section; iv) acquiring a fluorescence digital image of the tissue section; v) analyzing the brightfield digital image to obtain information for at least one tissue object; vi) analyzing the fluorescence digital image to obtain information for the same tissue objects as the brightfield digital image; and vii) aligning the data obtained from the brightfield and fluorescence digital images by matching the tissue objects identified in each image to identify at least one tissue object within the brightfield digital image that was not specifically stained for with the at least one chromogenic dye. The steps of analyzing and aligning are performed by a computer system configured to analyze digital images of tissue samples.

In the above embodiment, the steps of chromogenic staining, fluorescent staining, brightfield imaging, and fluorescence imaging can be performed in any logical order to obtain the disclosed invention. For example, the fluorescent staining may be followed immediately by the fluorescence imaging, and then the same tissue sample could be stained with the chromogenic dye and the brightfield image taken. Or the order could be reversed, such that chromogenic staining and brightfield imaging are followed by fluorescent staining and fluorescence imaging. It is also understood that the tissue sample could be stained with both chromogenic and fluorescent dyes, then both brightfield and fluorescence images could be acquired. However, it is understood that the illogical order of imaging before staining with appropriate dyes for that imaging modality is not part of the present invention.

In other embodiments, a dye that is visible in both imaging modalities, such as Fast Red, could be used to more easily identify tissue objects within both the brightfield and fluorescence digital images for alignment. A pair of dyes could also be used that stain for the same type of tissue objects, such as the cell nuclei, with one dye visible in each imaging modality, such as Hematoxylin and DAPI.

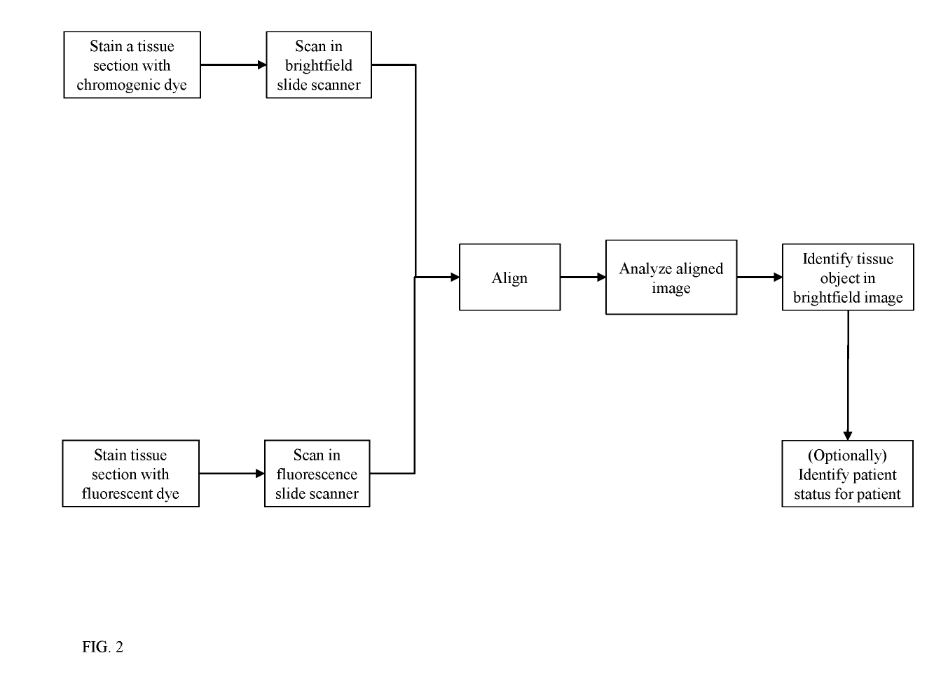

In another embodiment, the brightfield digital image and the fluorescence digital image can be aligned using common element between the two images and then the aligned image is analyzed to identify the tissue object in the brightfield digital image, as shown in FIG. 2. Many techniques exist for image-to-image alignment and are well known in the art. However, since the two images are of the same tissue slice, a rigid registration is typically sufficient. Allowing very small deformations can be beneficial because the brightfield and fluorescence images potentially have different local deformations caused by the imaging optics and, in the case of whole slide imaging, the tiling of individual fields of view.

In other embodiments, additional steps can be added to the previously disclosed method to allow for using the identified tissue objects to determine patient status for the patient from which the tissue sample was taken. This is performed by creating a score based on the identified tissue objects, then using that score and an established scoring scheme to determine that patient's status related to disease state, disease severity, disease progression, and/or therapy efficacy, along with other potential statuses related to diagnosis, prognosis, and treatment.

Additional embodiments include correcting for color cross-talk in the fluorescence digital image such that each color channel within the digital image contains information related to only a single dye. This additional step is particularly useful when multiple fluorescent dyes are present in the tissue sample, as the emission spectra of most fluorescent dyes have a long tail that can overlap with the emission of another fluorescent dye, thus contaminating the color channel associated with the overlapped dye.

One application of this method to identify tissue objects without directly staining the tissue object is in the use of computer aided diagnostics, such that a computer performs the diagnostic determination of the tissue sample for the patient. The core concept there is to teach the computer to distinguish specific tissue objects based on their morphology (their appearance in the brightfield image) while avoiding the need for a specific stain. This process requires lots of examples of the cells of interest, to allow the computer to generalize from these examples and be able to distinguish them in other settings. By automating how such examples are generated, this method reduces the need for hundreds, if not thousands, of hours of human pathologists to hand identify the tissue objects. Some tissue objects, such as specific immune cells, are notoriously difficult to identify without specific staining. The process thus is: (i) obtain tissue samples, prepared, imaged and analyzed as described in this invention, to derive a large set of example brightfield images of tissue objects stained with chromogenic dyes but not a dye that can be used to identify them; (ii) train a machine learning system the appearance of these cells; and (iii) apply the machine learning tool to brightfield images of tissue to identify those cells. Note that, by using the fluorescence modality to identify the cells of interest for the training set, the brightfield image appearance of these cells is not affected by the specific dye, which would introduce a bias in the machine learning tool.

What is claimed is:

1. A method comprising:
staining a tissue section with at least one chromogenic dye;
staining the tissue section with at least one fluorescent dye that stains for tissue objects;
acquiring a brightfield digital image of the tissue section;
acquiring a fluorescence digital image of the tissue section;
analyzing the brightfield digital image to obtain information for at least one tissue object;
analyzing the fluorescence digital image to obtain information for the at least one tissue object; and
matching the at least one tissue object from the brightfield digital image to the at least one tissue object from the fluorescence digital image to identify at least one tissue object within the brightfield digital image was not specifically stained for with the at least one chromogenic dye; and
wherein the aligning and analyzing steps are performed by a computer system configured to analyze digital images of tissue samples.

2. The method of claim 1, wherein the tissue object is stained with a dye that is visible in both the brightfield digital image and the fluorescence digital image.

3. The method of claim 2, wherein the dye that is visible in both the brightfield digital image and the fluorescence digital image is Fast Red.

4. The method of claim 1, wherein the tissue object is stained with a pair of dyes that stain a subset of tissue objects.

5. The method of claim 4, wherein the subset of tissue objects is a cellular compartment.

6. The method of claim 4, wherein the pair of dyes is Hematoxylin and DAPI.

7. The method of claim 1, wherein the fluorescent staining and fluorescence imaging are performed before the chromogenic staining and brightfield imaging.

8. The method of claim 1, wherein the chromogenic staining and brightfield imaging are performed before the fluorescent staining and fluorescence imaging.

9. The method of claim 1, wherein the chromogenic staining and fluorescent staining are performed before the brightfield and fluorescence imaging.

10. The method of claim 1, further comprising:
using the at least one identified tissue object to create an immune score; and
determining at least one patient status for a patient from whom the tissue section was acquired based on the immune score.

11. A method comprising:
staining a tissue section with at least one chromogenic dye;
staining the tissue section with at least one fluorescent dye that stains for tissue objects;
acquiring a brightfield digital image of the tissue section;
acquiring a fluorescence digital image of the tissue section;
aligning the brightfield digital image and the fluorescence digital image to create an aligned digital image using a common element for both the brightfield digital image and the fluorescence digital image; and
analyzing the aligned digital images using at least one fluorescent stain channel to identify at least one tissue object within the brightfield digital image that was not specifically stained for with the at least one chromogenic dye; and
wherein the aligning and analyzing steps are performed by a computer system configured to analyze digital images of tissue samples.

12. The method of claim 11, wherein the common element for both the brightfield digital image and the fluorescence digital image is a dye that is visible in both the brightfield digital image and the fluorescence digital image.

13. The method of claim 12, wherein the dye that is visible in both the brightfield digital image and the fluorescence digital image is Fast Red.

14. The method of claim 11, wherein the common element for both the brightfield digital image and the fluorescence digital image is a pair of dyes that stain a subset of tissue objects.

15. The method of claim 14, wherein the subset of tissue objects is a cellular compartment.

16. The method of claim 14, wherein the pair of dyes is Hematoxylin and DAPI.

17. The method of claim 11, wherein the fluorescent staining and fluorescence imaging are performed before the chromogenic staining and brightfield imaging.

18. The method of claim 11, wherein the chromogenic staining and brightfield imaging are performed before the fluorescent staining and fluorescence imaging.

19. The method of claim 11, wherein the chromogenic staining and fluorescent staining are performed before the brightfield and fluorescence imaging.

20. The method of claim 11, further comprising:
using the at least one identified tissue object to create an immune score; and
determining at least one patient status for a patient from whom the tissue section was acquired based on the immune score.
